(19) United States
(12) Patent Application Publication
(10) Pub. No.: US 2006/0078594 A1
(43) Pub. Date: Apr. 13, 2006

(54) Iron complex

(75) Inventors: Salomon Leendert Abrahamse, Vlaardingen (NL); Bernardus Nicodemus Van Buuren, Vlaardingen (NL); Werner Klaffke, Vlaardingen (NL); Willem Jan Kloots, Vlaardingen (NL)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco, Inc., d/b/a UNILEVER

(21) Appl. No.: 11/245,285

(22) Filed: Oct. 6, 2005

(30) Foreign Application Priority Data
Oct. 8, 2004 (EP) 04077948.0

(51) Int. Cl. A61K 47/00; A61K 31/555; A61K 31/295

(52) U.S. Cl. 424/439; 514/184; 514/502

(57) ABSTRACT

This invention relates to certain iron complexes which comprise ferrous or ferric iron and mono-, bi, tri- or hexadentate ligands containing a five- or six-membered aromatic or heterocyclic ring for use in the treatment and/or prevention of nutritional disorders and compositions containing such iron complexes. The iron complexes enhance the bioavailability of the iron and are particularly useful in the treatment and/or prevention of nutritional disorders, iron deficiency disorders and anaemia.

### TECHNICAL FIELD

This invention relates to certain iron complexes which comprise ferrous or ferric iron and mono-, bi-, tri- or hexadentate ligands containing a five- or six-membered aromatic or heterocyclic ring for use in the treatment and/or prevention of nutritional disorders; compositions containing such iron complexes; the use of such iron complexes in enhancing the bioavailability of iron and their use in the treatment and/or prevention of iron deficiency disorders and anaemia.

### BACKGROUND OF THE INVENTION

Ferrous (Fe (II)) and ferric (Fe(III)) iron are essential components in human nutrition since they are involved in many vital biological processes in the human body. For instance, iron is involved in the redox-active reaction centre of enzymes. It also represents the key co-ordination atom in haemoglobin and myoglobin which mediate the transport, storage and utilisation of oxygen in the body.

Iron deficiency disorders are amongst the most prevalent nutritional disorders throughout the world. According to the World Health Organisation [World Health Organisation Report of WHO/UNICEF/Joint Committee on Health Policy, 30th Session.JCHP30/95/4.5. Geneva] and other sources [see, for instance, Hurrell, Nutr. Rev. 55, 210-222 (1997)], 20 to 33% of the world's population, 50% in developing and 10% in developed countries, suffers from iron deficiency, mainly due to malnutrition. Anaemia in humans is mainly due to the lack or poor bioavailability of ferric or ferrous iron in the diet. Diets that are for various reasons high in vegetables and low in red meat may worsen the situation due to a high content of inositol phosphates and polyphenols. Iron deficiency also has consequences for child behaviour and development, work performance and immunity. Thus, the maintenance of sufficient iron levels is very important from a public health perspective.

Novel methods for delivering Fe(II) and Fe(III)-ions to the sites of intestinal absorption help to improve the iron status in humans and contribute to a healthier population. The fortification of foods with iron salts, such as ferrous sulphate or complexes is one such method. For instance, WO 01/67897 describes a fortified foodstuff comprising a fortifying amount of an inorganic compound prepared from sources of ferrous or ferric iron (e.g. ferrous sulphate), phosphate and ammonium, such as ferrous ammonium phosphate.

EDTA has been used in various foods and beverages. For instance, U.S. Pat. No. 4,299,853 describes the use of EDTA as a preservative for alcoholic beverages such as beer, wine and cider. U.S. Pat. No. 3,956,513 describes a solid product for use in the flavouring of food or beverages which contains a mixture of the disodium or dipotassium salt of EDTA with the tetra sodium or tetra potassium salt of EDTA. In U.S. Pat. No. 4,820,520, EDTA salts are described for use in combination with antiseptic agents to enhance the antifungal activity of the antiseptic agents in food and drinks. U.S. Pat. No. 4,937,085 describes a food preservation composition which prevents the discoloration of vegetables, such as potatoes, comprising citric acid, cysteine, ascorbic acid, and trace amounts of EDTA.

EDTA has also been used in the preparation of various iron-fortified foods and beverages. For instance, U.S. Pat. No. 5,667,825 and U.S. Pat. No. 5,534,275 describe the fortification of ready-to-eat cereal products with ferric EDTA as the iron source. U.S. Pat. No. 6,461,651 describes sodium-free iron (II) EDTA complexes which are useful for the preparation of iron-fortified processed foods. In addition, WO 03/013283 describes beverages and powdered beverage mixes fortified with ferric EDTA as the iron source. U.S. Pat. No. 4,020,158, U.S. Pat. No. 4,830,716 and U.S. Pat. No. 5,516,925 provide metal (including iron) amino acid chelates for administering to humans and other animals as a dietary supplement. In addition, WO 03/016332 discloses a method of enhancing the solubility of such iron amino acid chelates and iron proteinates. A food supplement comprising an iron amino acid chelate is marketed by Albion Laboratories, Inc. (Clearfield, Utah) under the tradename Ferrochel™. U.S. Pat. No. 5,653,987 describes a liquid pharmaceutical agent formulation suitable for oral or nasal delivery comprising a protein-based pharmaceutical agent, water and at least two absorption enhancing compounds which can include di-sodium EDTA.

A study of the oral application of organic ferrous salts was conducted in piglets to examine the antianaemic effect of iron oxalate, iron isophthalate, iron phthalate, iron terephthalate and iron nicotinate and to compare it with the antianaemic effect of iron fumarate (F. Kratky, “The peroral application of new organic iron compounds as a prophylactic measure against piglet physiological sideropenic anaemia”, Zivocisna Vyroba, (1972), 17(12), pp. 901-10). In the course of the trials, iron terephthalate and iron nicotinate showed similar antianaemic activity to iron fumarate.

WO 96/41627 and WO 02/24196 disclose iron complexes comprising iron in the ferric or ferrous state and a hydroxypyrone which may be used to increase the level of iron in a patient's bloodstream. Hydroxy-4-pyrones and 5-hydroxypyrones are preferred, especially 3-hydroxy-4-pyrones, such as maltol and ethylmaltol. WO 03/097627 also describes a method of forming such compounds which comprises reacting an iron salt of a carboxylic acid and a hydroxypyrone in an aqueous solution at a pH greater than 7.

WO 01/12163 discloses a nutritional supplement which comprises two different iron compounds, namely a rapidly dissolving iron compound and a slowly dissolving iron compound. The iron compounds can be selected from known iron compounds, such as iron (II) salts, iron (III) salts and particulate iron compositions.

It has been well reported that ferrous iron is more bioavailable than ferric iron, due to the more advantageous equilibria of the respective hydroxo-complexes at physiological pH values. Nevertheless, to date, no substance has been identified which satisfactorily enhances the bioavailability of iron whilst satisfying organoleptic requirements for foodstuffs, beverages and oral pharmaceutical formulations.

### SUMMARY OF THE INVENTION

In a first aspect, the present invention provides an iron complex comprising ferrous or ferric iron and a mono-, bi-, tri- or hexadentate ligand characterised in that the ligand is a compound of the general formula I 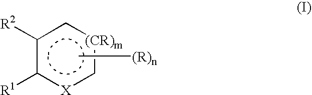
in which

m is 0 or 1;

n is 1 or 2;

X is CR or O;

each R independently represents a hydrogen atom, an oxo group, an optionally substituted alkyl group or a group —OR³, where R³ represents a hydrogen atom or an optionally substituted alkyl group;

R¹ represents an optionally substituted alkyl group or a group —CO—R⁴, where R⁴ represents a hydrogen atom or a group —OR³, where R³ is as defined above; and

R² represents a hydrogen atom or a group —OR³, where R³ is as defined above; with the proviso that, when X is O, then m is 0,

for use in the treatment and/or prevention of nutritional disorders.

In a second aspect, the invention provides an iron complex as defined above for use in medicine, especially for use in increasing the level of iron in a patient's bloodstream.

In a third aspect, the invention provides an iron complex as defined above for use in the treatment and/or prevention of iron deficiency disorders and/or anaemia.

In a fourth aspect, the invention provides the use of an iron complex as defined above for the manufacture of a medicament for use in the treatment and/or prevention of nutritional disorders.

In a fifth aspect, the invention provides the use of an iron complex as defined above for the manufacture of a medicament for use in increasing the level of iron in a patient's bloodstream.

In a sixth aspect, the invention provides the use of an iron complex as defined above for the manufacture of a medicament for use in the treatment and/or prevention of iron deficiency disorders and/or anaemia.

In a seventh aspect, the invention provides a food or beverage composition comprising an iron complex as defined above and a food acceptable or potable carrier.

In an eighth aspect, the invention provides an iron-fortified foodstuff or an iron-fortified beverage comprising a fortifying amount of an iron complex as defined above.

In a ninth aspect, the invention provides a diet supplement comprising an iron complex as defined above.

In a tenth aspect, the invention provides a pharmaceutical composition comprising an iron complex as defined above and a pharmaceutically acceptable carrier.

### DETAILED DESCRIPTION OF THE INVENTION

The present invention involves the development of an iron complex which is suitable for incorporation into foodstuffs, beverages, diet supplements and pharmaceutical compositions. The iron complexes comprise ferrous or ferric iron and bidentate ligands containing a five- or six-membered aromatic or heterocyclic ring. Specifically, the ligand may be a compound of the general formula I 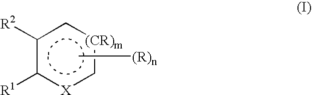
in which

m is 0 or 1;

n is 1 or 2;

X is CR or O;

each R independently represents a hydrogen atom, an oxo group, an optionally substituted alkyl group or a group —OR³, where R³ represents a hydrogen atom or an optionally substituted alkyl group;

R¹ represents an optionally substituted alkyl group or a group —CO—R⁴, where R⁴ represents a hydrogen atom or a group —OR³, where R³ is as defined above; and

R² represents a hydrogen atom or a group —OR³, where R³ is as defined above; with the proviso that, when X is O, then m is 0,

for use in the treatment and/or prevention of nutritional disorders.

Preferably, the ligand is a bidentate ligand.

Any alkyl group, unless otherwise specified, may be linear or branched and may contain up to 12, preferably up to 6, and especially up to 4, carbon atoms. Preferred alkyl groups are methyl, ethyl, propyl and butyl, particularly methyl and ethyl, and especially methyl. When an alkyl moiety forms part of another group, for example the alkyl moiety of an alkoxy group, it is preferred that it contains up to 6, especially up to 4, carbon atoms. Preferred alkyl moieties are methyl and ethyl, especially methyl.

When any of the foregoing substituents are designated as being optionally substituted, the substitutent groups which are optionally present may be any one or more of those customarily employed in the development of foodstuffs, beverages and/or pharmaceutical compounds and/or the modification of such compounds to influence their structure/activity, stability, bioavailability or other property. Specific examples of such substitutents include, for example, halogen atoms, nitro, hydroxyl, alkyl, haloalkyl, alkoxy, haloalkoxy, amino, alkylamino, dialkylamino, formyl, alkoxycarbonyl, carboxyl and alkanoyl groups.

When any of the foregoing optional substituents represents or contains an alkyl substituent group, this may be linear or branched and may contain up to 6, preferably up to 4, carbon atoms. Preferred optional substituents include halogen atoms, hydroxyl, C₁₋₄ alkyl, C₁₋₄ haloalkyl, C₁₋₄ alkoxy, C₁₋₄ haloalkoxy, amino, C₁₋₄ alkylamino and di-(C₁₋₄ alkyl)amino groups. Particularly preferred optional substituents include halogen atoms, hydroxyl, C₁₋₄ alkyl, C₁₋₄ alkoxy and amino groups, with halogen atoms and hydroxyl groups being especially preferred.

It is preferred that R¹ represents a C₁₋₄ alkyl group, especially an ethyl or, more preferably, a methyl group, or a group —CO—R⁴. More preferably, R¹ represents a group —CO—R⁴. Preferably, R⁴ represents a hydrogen atom, a hydroxyl group or a C₁₋₄ alkoxy group. More preferably, R⁴ represents a hydrogen atom or a hydroxyl, methoxy or ethoxy group.

X can be CR or O. Thus, when X is CR, all positions of the five- or six-membered ring may be substituted. However, it is also possible for only one of the positions of the five- or six-membered ring to be substituted or for only some of these positions to be substituted. When X is O, the O-atom is not substituted but one, some or all of the remaining ring atoms may be substituted.

Although m can be 0 or 1, when X is CR, it is preferred that m is 1.

It is preferred that R² represents a hydrogen atom or a hydroxyl or C₁₋₄ alkoxy group. More preferably, R² represents a hydrogen atom or a hydroxyl or methoxy group, especially a hydroxyl group.

Preferably, each R independently represents a hydrogen atom or a hydroxyl, C₁₋₄ alkyl or C₁₋₄ alkoxy group. More preferably, each R independently represents a hydrogen atom or a hydroxyl, methyl or methoxy group.

When X is O, R may also represent an oxo group. Compounds in which the oxo group is located at the 2- or 3-position, that is, furan-2-ones and furan-3-ones, are particularly preferred, especially furan-3-ones.

In a preferred sub-group of compounds, m is 1, n is 1, X is CR, each R independently represents a hydrogen atom or a hydroxyl or methoxy group, R¹ is a group —CO—R⁴ where R⁴ represents a hydrogen atom or a hydroxyl group, especially a hydrogen atom, and R² represents a hydrogen atom or a hydroxyl group. Di- or tri-substituted phenyl groups are particularly preferred. Preferred substitution patterns include 1,2-, 1,2,3-, 1,2,4-, 1,3,4- and 1,2,5-substitution of the phenyl ring. Compounds having a hydroxyl group ortho to the group —CO—R⁴ are especially preferred.

A particularly preferred sub-group of compounds, includes 2-hydroxybenzaldehyde, 2-hydroxy-3-methoxybenzaldehyde (o-vanillin), 2,5-dihydroxybenzaldehyde, 2-hydroxy-4-methoxybenzaldehyde and 3-hydroxy-4-methoxybenzoic acid.

In another preferred sub-group of compounds, m is 0, n is 1, X is O, R represents a hydrogen atom, an oxo group or a methyl or ethyl group, R¹ is a methyl group or a group —CO—R⁴ where R⁴ represents a hydrogen atom, and R² represents a hydrogen atom. Di-substituted furan groups are particularly preferred. The preferred substitution pattern is 2,5-substitution.

A particularly preferred sub-group of compounds includes 5-methyl-2-furaldehyde, 2-ethyl-4-hydroxy-5-methyl-3(2H)-furanone, 2,5-dimethyl-4-hydroxy-3(2H)-furanone and 4-hydroxy-5-methyl-3(2H)-furanone.

It is particularly advantageous that the iron complex is soluble in water.

The ferrous or ferric iron can be provided from any suitable source. The ferrous iron source can be any food or pharmaceutical grade ferrous salt, such as ferrous sulphate, ferrous ammonium sulphate, ferrous chloride, ferrous malate, ferrous acetate, ferrous gluconate, ferrous nitrate, ferrous lactate, ferrous fumarate, ferrous succinate, ferrous oxide, ferrous hydroxide or mixtures thereof. Ferrous sulphate is particularly preferred.

The ferric iron source can be any food or pharmaceutical grade ferric salt, such as ferric sulphate, ferric chloride, ferric nitrate, ferric acetate, ferric malate, ferric ammonium acetate, ferric formate, ferric oxide, ferric hydroxide or mixtures thereof. Ferric sulphate is particularly preferred.

An iron complex as defined above can be prepared by a method which comprises reacting a ferrous or ferric iron salt with a compound of the general formula I as defined above in solution. Preferably, the molar ratio of the iron salt to the compound of general formula I is from 1:1 to 1:6, more preferably from 1:2 to 1:3, especially 1:2.

The iron complexes-as defined above are useful for enhancing the bioavailability of iron. It is therefore convenient to administer the iron complexes of the invention in the form of compositions. The invention thus also provides a composition comprising an iron complex as defined above and a carrier.

The composition may be a food composition in which case the carrier may be a food acceptable carrier. Suitable food acceptable carriers include proteins, fats, starches, sugars and the like. The composition may be a beverage composition in which case the carrier may be a potable carrier. Suitable potable carriers include water, milk and other appropriate liquids. Beverage compositions include ready-to-drink beverages and powdered beverage mixes which can be reconstituted using appropriate liquids.

The invention also includes an iron-fortified foodstuff which comprises a fortifying amount of an iron complex as defined above and an iron-fortified beverage which comprises a fortifying amount of an iron complex as defined above and a potable liquid. Such iron-fortified beverages include ready-to-drink beverages as well as powdered beverage mixes. Preferred foodstuffs include cereals and flours and products made from cereals and/or flours, such as porridges and nutritional bars. Dairy products, such as milk products, are also preferred. Food products for invalids and the elderly may also be iron-fortified in accordance with the invention.

The amount of iron in such food or beverage compositions may be from 1 to 200 ppm, preferably from 5 to 100 ppm and more preferably from 10 to 75 ppm. Ideally, the amount of iron should be such that the recommended daily allowance (RDA) of 40 mg per day can be reached with no more than 3 servings a day.

The invention further includes a diet supplement which comprises an iron complex as defined above. Such diet supplements may be administered in isolation or added to foodstuffs or beverages.

Compositions of the invention may also be in the form of a pharmaceutical composition in which case the carrier may be a pharmaceutically acceptable carrier.

A pharmaceutically acceptable carrier may be any material with which the iron complex is formulated to facilitate administration. A carrier may be a solid or a liquid, including a material which is normally gaseous but which has been compressed to form a liquid, and any of the carriers normally used in formulating pharmaceutical compositions may be used. Preferably, compositions according to the invention contain 0.5 to 95% by weight of active ingredient.

The iron complexes of the invention can be formulated as, for example, tablets, capsules, suppositories or solutions. These formulations can be produced by known methods using conventional solid carriers such as, for example, lactose, starch or talcum or liquid carriers such as, for example, water, fatty oils or liquid paraffins. Other carriers which may be used include materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminium silicates; and amino acids having from 2 to 12 carbon atoms such as a glycine, L-alanine, L-aspartic acid, L-glutamic acid, L-hydroxyproline, L-isoleucine, L-leucine and L-phenylalanine.

Auxiliary components such as tablet disintegrants, solubilisers, preservatives, antioxidants, surfactants, viscosity enhancers, colouring agents, flavouring agents, pH modifiers, sweeteners or taste-masking agents may also be incorporated into the composition. Suitable colouring agents include red, black and yellow iron oxides and FD & C dyes such as FD & C blue No. 2 and FD & C red No. 40 available from Ellis & Everard. Suitable flavouring agents include mint, raspberry, liquorice, orange, lemon, grapefruit, caramel, vanilla, cherry and grape flavours and combinations of these. Suitable pH modifiers include sodium hydrogencarbonate, citric acid, tartaric acid, phosphoric acid, hydrochloric acid and maleic acid. Suitable sweeteners include aspartame, acesulfame K and thaumatin. Suitable taste-masking agents include sodium hydrogencarbonate, ion-exchange resins, cyclodextrin inclusion compounds, adsorbates or microencapsulated actives.

As mentioned above, the iron complexes as defined above have been found to enhance the bioavailability of iron and increase the level of iron in a patient's bloodstream. Thus, the present invention also provides an iron complex as defined above for use in medicine, particularly for the treatment and/or prevention of nutritional disorders, preferably iron deficiency disorders and, especially, anaemia.

The invention also includes the use of an iron complex as defined above for the manufacture of a medicament for use in the treatment and/or prevention of nutritional disorders, particularly iron deficiency disorders and, especially, anaemia. The invention further provides the use of an iron complex as defined above for the manufacture of a medicament for use in increasing the level of iron in a patient's bloodstream.

In another aspect, the invention provides a method of treating or preventing nutritional disorders, particularly iron deficiency disorders and, especially, anaemia which comprises administering to a patient a therapeutically or prophylactically effective amount of an iron complex as defined above.

The invention is further illustrated by the following examples.

### EXAMPLES

Materials and Methods

Chemicals were obtained from Sigma-Aldrich. Water was deionised and filtered (Milli-Q®).

Measuring Iron Complexation with Natural Compounds

One volume of a solution of 0.1 mM FeSO₄ was mixed with one volume of a solution containing 0.3 mM of the potential iron complexing agent. Subsequently, one volume of 0.2 mM terpyridine solution was added. The terpyridine-iron complex results in a red coloured solution with an absorption maximum at 555 nm wavelength. To simulate the pH range of the intestinal tract, the assay was performed at pH7, pH4.5 and pH 2.

Preparation of the Complexes

To a solution of FeSO₄ (0.899 mol) in deionised (Milli-Q®) and degassed water (680 mL) was added a threefold molar excess (2.7 mol) of the respective complexing agent dissolved in sufficient absolute ethanol under a nitrogen atmosphere under constant stirring. Spontaneous complex formation could be observed by rapid colour change from a slight green to a darker green/green-red colour.

Preparation of the Dough

The flour (100 g, Pelikan, Meneba, NL) was mixed gently with the previously prepared solution (68 mL) until a homogenous dough was obtained. The crude mass was aliquoted and small lumps were formed (10 g±0.5 g) which were used for the iron dialysability assay.

Iron Dialysability Assay

All glassware and paddles were cleaned with 10% HNO₃ (24 hours) and rinsed five times with Milli-Q® water. 1 g of dough was homogenised in 40 ml Milli-Q® water with a Braun Blender Type 4142, at level 1 for 1 s, level 2 for 1s, and level 3 for 10s. The resulting suspension was poured into a VanKel Vial and the blender beaker was rinsed twice with Milli-Q® water (20 mL) and the rinse water was added to the suspension. The stirring speed of the paddle was adjusted to 200 rpm at 37° C. (VanKel VK700, Varian). The pH of the suspension was adjusted to 2.0 with 6N HCl and a Pepsin-HCl solution (5 mL) was added. After an incubation time of 15 minutes, the pH was adjusted with 0.5N NaOH to 2.0 and the suspension filled up to 90 mL with Milli-Q® water. Stirring was maintained for 105 minutes. Three homogenised aliquots of about 20 g each were sampled, (i) one of which was stored at −20° C., (ii) another was added to 5 ml pancreatic-bile solution and adjusted to pH 7.5 with 0.5N NaOH, and (iii) the third was transferred into an Erlenmeyer flask (20 mL). Sample (ii) was incubated for 30 minutes and re-adjusted (if necessary) to pH 7.5 with 0.5N NaOH, the amount of NaOH was used to determine the amount of NaCO₃ needed to adjust the pH of the simulated gastric solution to pH 7.5. A clean dialysis membrane (20 cm, Spectra/Por7, Molecular weight cut-off 8 kD) was filled with a determined amount of 0.1M NaCO₃ solution and adjusted to a total volume of 25 mL with Milli-Q® water. The dialysis bag was placed in the Erlenmeyer flask containing aliquot (iii), and shaken at 37° C. at 100 beats/min for 30 minutes while the pH was being monitored. Upon completion, 5 ml pancreatic/bile-extract were added to the flask, and agitation was continued for 2 hours. After that time, the contents of the dialysis bag were recovered for quantitative analysis of the iron content.

Measurement of Total Iron Content

Total iron content of yeast and wheat flour was determined by inductively coupled plasma atomic emission spectrometry. Briefly, samples were digested in 5 ml 65% nitric acid and 0.5 ml 30% hydrogen peroxide in closed vessels in a microwave oven at high temperature and high pressure (110 bar). After digestion, the volume was adjusted to 50 ml using demineralised water and sprayed into the inductively coupled plasma of a plasma emission spectrometer (Perkin Elmer 3300 DV Inductive Coupled Plasma-Optical Emission Spectrometer). The emission of the individual elements was measured at specific wavelengths and concentrations were quantified from standard solutions.

Measurement of Ionic Iron in Dialysate

Dialysable ionic iron (sum of Fe²⁺ and Fe³⁺) was determined using a Roche/Hitachi Analyser and reagents for the analysis of iron in human serum based on FerroZine® Iron Reagent (i.e. 3-(2-pyridyl)-5,6-diphenyl-1,2,4-triazine-p,p′-disulphonic acid, monosodium salt hydrate). The analyses were performed according to the instructions of the supplier of the reagents (Roche Diagnostics Nederland BV), using the dialysate of the in vitro iron availability assay instead of serum.

### EXAMPLES

Iron Complexation

Table 1. Iron-binding characteristics of molecules.

The molecules were evaluated for solubility and iron complexation at pH 7.0, 4.5 and 2.0. Values are relative absorbance at 555 nm wavelength against sodium iron EDTA. The higher the value, the stronger the binding of iron to the indicated compound and the better the complexing agent. Data are the results from single measurements.

| | Solubility/Complexation | | |
|---|---|---|---|
| Compound | pH 7 | pH 4.5 | pH 2 |
| 2-hydroxy benzaldehyde | 0.94 | 0.37 | 0.53 |
| 2-hydroxy-3-methoxy benzaldehyde (o- | 2.19 | 0.39 | 0.60 |
| vanillin) | | | |
| 5-methoxy-2-furaldehyde | 1.24 | 0.61 | 0.63 |
| 2,5-dihydroxybenzaldehyde | 1.70 | 0.38 | 0.89 |
| 2-hydroxy-4-methoxybenzaldehyde | 1.89 | 0.46 | 0.62 |
| 3-hydroxy-4-methoxybenzoic acid | 0.88 | 0.61 | 0.79 |
| 2-ethyl-4-hydroxy-5-methyl-3(2H)- | 0.87 | 0.59 | 1.06 |
| furanone | | | |
| 2,5-dimethyl-4-hydroxy-3(2H)-furanone | 0.81 | 0.65 | 1.04 |
| 4-hydroxy-5-methyl-3(2H)-furanone | 0.89 | 0.46 | 1.01 |

Iron Dialysability From Food

TABLE 2
The iron content and percentage of in vitro dialysable iron were
calculated and used to calculate the relative iron availability of dough
from whole-grain wheat flour fortified with 50 mg/kg iron from different
compounds, as indicated.
Results are means ± SD of 4-5 experiments.

| | Relative |
|---|---|
| Iron compounds | availability* |
| Iron sulphate | 1.0 |
| Iron 2-hydroxybenzaldehyde | 1.5** |
| Iron 2-hydroxy-3-methoxy benzaldehyde (o-vanillin) | 2.4** |
| Iron 5-methyl-2-furaldehyde | 1.3 |
| Iron 2,5-dihydroxybenzaldehyde | 1.8** |
| Iron 2-hydroxy-4-methoxy benzaldehyde | 1.1 |
| Iron 3-hydroxy-4-methoxy benzoic acid | 1.0 |
| Iron 2-ethyl-4-hydroxy-5-methyl-3(2H)-furanone | 5.0** |
| Iron 2,5-dimethyl-4-hydroxy-3(2H)-furanone | 1.3 |
| Iron 4-hydroxy-5-methyl-3(2H)-furanone | 1.3 |

*Normalized relative to the iron availability of dough made from whole-grain wheat flour fortified with ferrous sulfate.
**Significantly different from ferrous sulfate (ANOVA, p < 0.05).

1. A method of treating and/or preventing nutritional disorders which comprises administering to a patient a therapeutically and/or prophylactically effective amount of an iron complex comprising ferrous or ferric iron and a mono-, bi-, tri- or hexadentate bidentate ligand characterised in that the ligand is a compound of the general formula I 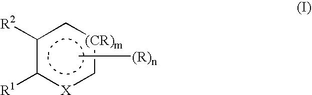
in which
m is 0 or 1;
n is 1 or2;
X is CR or O;
each R independently represents a hydrogen atom, an oxo group, an optionally substituted alkyl group or a group —OR³, where R³ represents a hydrogen atom or an optionally substituted alkyl group;
R¹ represents an optionally substituted alkyl group or a group —CO—R⁴, where R⁴ represents a hydrogen atom or a group —OR³, where R³ is as defined above; and
R² represents a hydrogen atom or a group —OR³, where R³ is as defined above;
with the proviso that, when x is O, then m is 0:

2. A method according to claim 1 in which the ligand is a bidentate ligand.

3. A method according to claim 1 in which R¹ represents a C₁₋₄ alkyl group or a group —CO—R⁴.

4. A method according to claim 1 in which R¹ represents a group —CO—R⁴.

5. A method according to claim 1 in which R⁴ represents a hydrogen atom, a hydroxyl group or a C₁₋₄ alkoxy group.

6. A method according to claim 1 in which R² represents a hydrogen atom or a hydroxyl or C₁₋₄ alkoxy group.

7. A method according to claim 1 in which each R independently represents a hydrogen atom or a hydroxyl, C₁₋₄ alkyl or C₁₋₄ alkoxy group.

8. A method according to claim 1 in which X is CR.

9. A method according to claim 1 in which m is 1.

10. A method according to claim 1 in which X is O.

11. A method according to claim 1 in which n is 1.

12. A method according to claim 1 which is soluble in water.

13. A method of increasing the level of iron in a patient's bloodstream which comprises administering to a patient a therapeutically and/or prophylactically effective amount of an iron complex comprising ferrous or ferric iron and a mono-, bi-, tri- or hexadentate bidentate ligand characterised in that the ligand is a compound of the general formula I 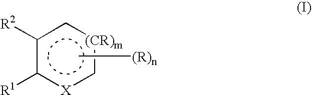
in which
m is 0 or 1;
n is 1 or 2;
X is CR or O;
each R independently represents a hydrogen atom, an oxo group, an optionally substituted alkyl group or a group —OR³, where R³ represents a hydrogen atom or an optionally substituted alkyl group;
R¹ represents an optionally substituted alkyl group or a group —CO—R⁴, where R⁴ represents a hydrogen atom or a group —OR³, where R³ is as defined above; and
R² represents a hydrogen atom or a group —OR³, where R³ is as defined above;
with the proviso that, when X is O, then m is 0.

14. A method of treating and/or preventing iron deficiency disorders which comprises administering to a patient a therapeutically and/or prophylactically effective amount of an iron complex comprising ferrous or ferric iron and a mono-, bi-, tri- or hexadentate bidentate ligand characterised in that the ligand is a compound of the general formula I 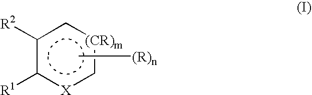
in which
m is 0 or 1;
n is 1 or2;
X is CR or O;
each R independently represents a hydrogen atom, an oxo group, an optionally substituted alkyl group or a group —OR³, where R³ represents a hydrogen atom or an optionally substituted alkyl group;
R¹ represents an optionally substituted alkyl group or a group —CO—R⁴, where R⁴ represents a hydrogen atom or a group —OR³, where R³ is as defined above; and
R² represents a hydrogen atom or a group —OR³, where R³ is as defined above;
with the proviso that, when X is O, then m is 0.

15. A method of treating and/or preventing anaemia which comprises administering to a patient a therapeutically and/or prophylactically effective amount of an iron complex comprising ferrous or ferric iron and a mono-, bi-, tri- or hexadentate bidentate ligand characterised in that the ligand is a compound of the general formula I 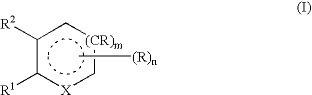
in which
m is 0 or 1;
n is 1 or2;
X is CR or O;
each R independently represents a hydrogen atom, an oxo group, an optionally substituted alkyl group or a group —OR³, where R³ represents a hydrogen atom or an optionally substituted alkyl group;
R¹ represents an optionally substituted alkyl group or a group —CO—R⁴, where R⁴ represents a hydrogen atom or a group —OR³, where R³ is as defined above; and
R² represents a hydrogen atom or a group —OR³, where R³ is as defined above;
with the proviso that, when X is O, then m is 0.

16. A food composition comprising a food acceptable carrier and an iron complex comprising ferrous or ferric iron and a mono-, bi-, tri- or hexadentate bidentate ligand characterised in that the ligand is a compound of the general formula I 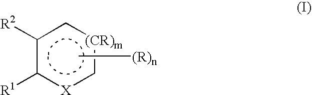
in which
m is 0 or 1;
n is 1 or 2;
X is CR or O;
each R independently represents a hydrogen atom, an oxo group, an optionally substituted alkyl group or a group —OR³, where R³ represents a hydrogen atom or an optionally substituted alkyl group;
R¹ represents an optionally substituted alkyl group or a group —CO—R⁴, where R⁴ represents a hydrogen atom or a group —OR³, where R³ is as defined above; and
R² represents a hydrogen atom or a group —OR³, where R³ is as defined above; with the proviso that, when X is O, then m is 0.

17. An iron-fortified foodstuff comprising a fortifying amount of an iron complex comprising ferrous or ferric iron and a mono-, bi-, tri- or hexadentate bidentate ligand characterised in that the ligand is a compound of the general formula I 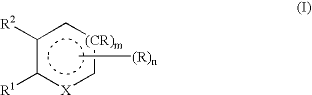
in which
m is 0 or 1;
n is 1 or2;
X is CR or O;
each R independently represents a hydrogen atom, an oxo group, an optionally substituted alkyl group or a group —OR³ where R³ represents a hydrogen atom or an optionally substituted alkyl group;
R¹ represents an optionally substituted alkyl group or a group —CO—R⁴, where R⁴ represents a hydrogen atom or a group —OR³ where R³ is as defined above; and
R² represents a hydrogen atom or a group —OR³, where R³ is as defined above; with the proviso that, when X is O, then m is 0.

18. A beverage composition comprising a potable carrier and an iron complex comprising ferrous or ferric iron and a mono-, bi-, tri- or hexadentate bidentate ligand characterised in that the ligand is a compound of the general formula I 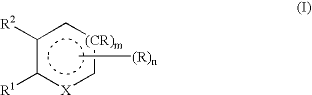
in which
m is 0 or 1;
n is 1 or 2;
X is CR or O;
each R independently represents a hydrogen atom, an oxo group, an optionally substituted alkyl group or a group —OR³, where R³ represents a hydrogen atom or an optionally substituted alkyl group;
R¹ represents an optionally substituted alkyl group or a group —CO—R⁴, where R⁴ represents a hydrogen atom or a group —OR³, where R³ is as defined above; and
R² represents a hydrogen atom or a group —OR³, where R³ is as defined above;
with the proviso that, when X is O, then m is 0.

19. An iron-fortified beverage comprising a potable liquid and a fortifying amount of an iron complex comprising ferrous or ferric iron and a mono-, bi-, tri- or hexadentate bidentate ligand characterised in that the ligand is a compound of the general formula I 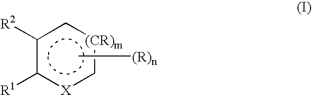
in which
m is 0 or 1;
n is 1 or 2;
X is CR or O;
each R independently represents a hydrogen atom, an oxo group, an optionally substituted alkyl group or a group —OR³, where R³ represents a hydrogen atom or an optionally substituted alkyl group;
R¹ represents an optionally substituted alkyl group or a group —CO—R⁴, where R⁴ represents a hydrogen atom or a group —OR³, where R³ is as defined above; and
R² represents a hydrogen atom or a group —OR³, where R³ is as defined above;
with the proviso that, when X is O, then m is 0.

20. A diet supplement comprising an iron complex comprising ferrous or ferric iron and a mono-, bi-, tri- or hexadentate bidentate ligand characterised in that the ligand is a compound of the general formula I 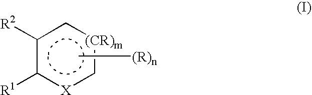
in which
m is 0 or 1;
n is 1 or2;
X is CR or O;
each R independently represents a hydrogen atom, an oxo group, an optionally substituted alkyl group or a group —OR³, where R³ represents a hydrogen atom or an optionally substituted alkyl group;
R¹ represents an optionally substituted alkyl group or a group —CO—R⁴, where R⁴ represents a hydrogen atom or a group —OR³, where R³ is as defined above; and
R² represents a hydrogen atom or a group —OR³, where R³ is as defined above;
with the proviso that, when X is O, then m is 0.

21. A pharmaceutical composition comprising a pharmaceutically acceptable carrier and an iron complex comprising ferrous or ferric iron and a mono-, bi-, tri- or hexadentate bidentate ligand characterised in that the ligand is a compound of the general formula I 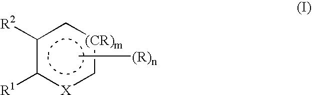
in which
m is 0 or 1;
n is 1 or 2;
X is CR or O;
each R independently represents a hydrogen atom, an oxo group, an optionally substituted alkyl group or a group —OR³, where R³ represents a hydrogen atom or an optionally substituted alkyl group;
R¹ represents an optionally substituted alkyl group or a group —CO—R⁴, where R⁴ represents a hydrogen atom or a group —OR³, where R³ is as defined above; and
R² represents a hydrogen atom or a group —OR³, where R³ is as defined above; with the proviso that, when X is O, then m is 0.
